(19) United States
(12) Patent Application Publication
(10) Pub. No.: US 2024/0342443 A1
(43) Pub. Date: Oct. 17, 2024

(54) LYMPHATICS PUNCTURE NEEDLE FOR ACCESS AND STABILIZATION

(71) Applicant: Ghassan S. Kassab, La Jolla, CA (US)

(72) Inventor: Ghassan S. Kassab, La Jolla, CA (US)

(21) Appl. No.: 18/439,306

(22) Filed: Feb. 12, 2024

Related U.S. Application Data
(63) Continuation of application No. 16/870,332, filed on May 8, 2020, now abandoned.
(60) Provisional application No. 62/845,677, filed on May 9, 2019.

(51) Int. Cl. A61M 25/06; A61M 25/01; A61M 25/09

(52) U.S. Cl. CPC A61M 25/06; A61M 25/0136; A61M 25/0138; A61M 2025/0166; A61M 2025/0681; A61M 2025/09133

(57) ABSTRACT

A lymphatic puncture device with a stabilization mechanism preventing overperforation of the lymph node is described. In some embodiments, the device comprises flared tines and a retractable sleeve and a needle extending distal beyond the flared tines. Upon retraction of the sleeve and exposure of the flared tines, the needle can be inserted into the lymph node without risk of overperforation.

### PRIORITY

The present application is related to, claims the priority benefit of, and is a U.S. continuation patent application of, U.S. patent application Ser. No. 16/870,332, filed May 9, 2020, which is related to, and claims the priority benefit of, U.S. Provisional Patent Application Ser. No. 62/845,677, filed on May 9, 2019, the contents of which are hereby incorporated by reference in their entirety into this disclosure.

### BACKGROUND

Medical professionals may need to puncture a lymph node for var10us medical procedures or applications, such as biopsies, lymphangiograms, or lymphatic interventions. However, puncturing a lymph node is very tricky due to the lymph structure's complex geometry and relatively thin walls. These complications make lymph node puncture access quite difficult for physicians or surgeons. Typically, medical professionals will use ultrasound guidance to first visualize the lymph nodes, and then the puncture needle will be guided into the lymph node structure. However, even with the assistance of ultrasound guidance, there is still a risk of over-perforation of the lymph node or loss of access given the ‘pancake’ or sinusoid shaped nature of the lymph node structure.

Thus, there is a significant medical need for a lymphatics puncture needle with a stabilization mechanism that not only ensures access to the lymph node, but also retention of that access while preventing over-perforation.

### SUMMARY OF THE INVENTION

In one embodiment, a device for puncturing a lymph node comprises: a sleeve having a proximal portion and a distal portion and a lumen extending between the proximal and distal portions; an engagement catheter disposed within the lumen of the sleeve and having a proximal end and a distal end and a lumen extending between the proximal and distal ends; a flared stabilizer disposed at the distal end of the engagement catheter and flaring away from a center longitudinal axis of the engagement catheter, wherein the flared stabilizer is operably deployed by retraction of the sleeve toward its proximal portion; and a lymphatics puncture needle disposed within the lumen of the engagement catheter and operable to puncture a lymph node.

In one embodiment of a device for puncturing a lymph node, the flared stabilizer further comprises a plurality of flared tines extending outwardly from a center longitudinal axis of the flared stabilizer on the engagement catheter.

In one embodiment of a device for puncturing a lymph node, the plurality of flared tines comprises four axially cut tines.

In one embodiment of a device for puncturing a lymph node, the plurality of flared tines comprises tines extending 1-3 mm.

In one embodiment of a device for puncturing a lymph node, the lymphatics needle comprises a 23-25G distal to 21-22G proximal needle.

In one embodiment of a device for puncturing a lymph node, the lymphatic puncture needle is inserted 3-5 mm into the lymph node before the flared stabilizer contacts an outer wall surface of the lymph node, preventing over-perforation of the lymph node.

In one embodiment of a device for puncturing a lymph node, the lymphatics puncture needle has a length of 3-5 mm, distal to the flared stabilizer.

In one embodiment of a device for puncturing a lymph node, the lymphatics puncture needle is coated with a material to aid in ultrasound visualization.

In one embodiment, the invention comprises a method of puncturing a lymph node, comprising: advancing a lymphatic puncture needle toward a lymph node; retracing a sleeve to deploy a flared stabilizer; advancing the lymphatic puncture needle to puncture the lymph node, until the flared stabilizer stops against an outer wall surface of a lymph node; applying gentle pressure to the lymphatic needle to maintain a puncture site and stability without over-perforation of the lymph node.

In one embodiment of a method of puncturing a lymph node, the step of advancing the lymphatics puncture needle further comprises advancing the needle under ultrasound guidance.

In one embodiment of a method of puncturing a lymph node, the flared stabilizer further comprises a plurality of tines extending outwardly from a center longitudinal axis of the flared stabilizer.

In one embodiment of a method of puncturing a lymph node, the plurality of flared tines comprises four axially cut tines.

In one embodiment of a method of puncturing a lymph node, the step of retracting the sleeve comprises pulling the sleeve toward the operating physician.

In one embodiment of a method of puncturing a lymph node, advancing the lymphatic puncture needle comprises advancing the needle 3-5 mm, until the flared stabilizer stops against an outer wall surface of a lymph node to prevent over-perforation of the lymph node.

In one embodiment of a method of puncturing a lymph node, the step of retracting the sleeve comprises operating an actuator located on a handle of a catheter.

In one embodiment, a system for puncturing and stabilizing a lymph node, comprises: a sleeve having a proximal portion and a distal portion and a lumen extending between the proximal and distal portions; an engagement catheter disposed within the lumen of the sleeve and having a proximal end and a distal end and a lumen extending between the proximal and distal ends; a flared stabilizer disposed at the distal end of the engagement catheter and flaring away from a center longitudinal axis of the engagement catheter, a lymphatics puncture needle disposed within the lumen of the engagement catheter and operable to puncture a lymph node; and wherein the flared stabilizer is operably deployed by retraction of the sleeve toward its proximal portion to expose a plurality of flared tines which physically contact an outer wall surface of a lymph node to stop further advancement of the lymphatics needle to prevent over-perforation of the lymph node.

In one embodiment of a system for puncturing and stabilizing a lymph node, the plurality of flared tines comprises four axially cut tines.

In one embodiment of a system for puncturing and stabilizing a lymph node, the plurality of flared tines comprises tines extending 1-3 mm.

In one embodiment of a system for puncturing and stabilizing a lymph node, the lymphatics needle comprises a 23-25G distal to 21-22G proximal needle.

In one embodiment of a system for puncturing and stabilizing a lymph node, the lymphatic puncture needle is inserted 3-5 mm into the lymph node before the flared stabilizer contacts an outer wall surface of the lymph node, preventing over-perforation of the lymph node.

In one embodiment of a system for puncturing and stabilizing a lymph node, the lymphatics puncture needle has a length of 3-5 mm, distal to the flared stabilizer.

In one embodiment of a system for puncturing and stabilizing a lymph node, the lymphatics puncture needle has a contrast coating to aid visualization under ultrasound guidance.

### BRIEF DESCRIPTION OF THE DRAWINGS

The disclosed embodiments and other features, advantages, and disclosures contained herein, and the matter of attaining them, will become apparent and the present disclosure will be better understood by reference to the following description of various exemplary embodiments of the present disclosure taken in conjunction with the accompanying drawings, wherein:

FIG. 1 illustrates a perspective view of a first embodiment of a lymphatic puncture needle for puncture access and stabilization;

FIG. 2 illustrates a perspective view of an embodiment of a lymphatic puncture needle within a needle sheath approaching the lymph node in preparation for puncturing the lymph node;

FIG. 3 illustrates a perspective view of an embodiment of a lymphatic puncture needle with the needle sheath retracted to deploy or expose the flared stopper or stabilizer;

FIG. 4 illustrates a perspective view of an embodiment of a lymphatic puncture needle with the flared stopper or stabilizer in contact with an outer wall surface of the lymph node and the puncture needle inserted within the lymph node; and

FIG. 5 illustrates a flowchart of steps of an exemplary operation of a lymphatic puncture needle.

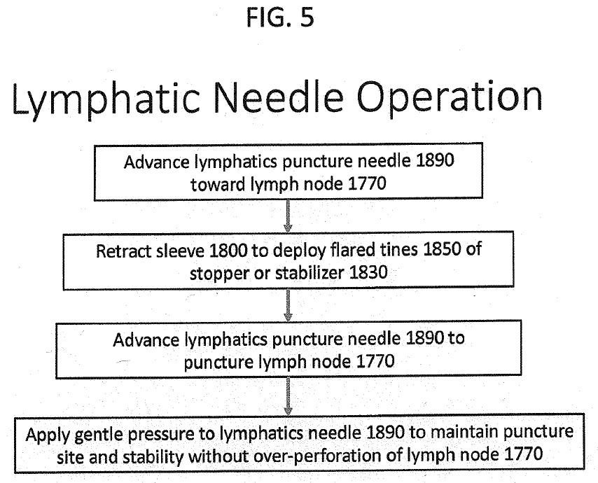

As such, an overview of the features, functions and/or configurations of the components depicted in the various figures will now be presented. It should be appreciated that not all of the features of the components of the figures are necessarily described and some of these non-discussed features (as well as discussed features) are inherent from the figures themselves. Other non-discussed features may be inherent in component geometry and/or configuration. Furthermore, wherever feasible and convenient, like reference numerals are used in the figures and the description to refer to the same or like parts or steps. The figures are in a simplified form and not to precise scale.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

The present disclosure includes various engagement catheters 1810 having a lymphatics needle 1890 for puncture access and stabilization, and systems and methods for operating the engagement catheters to puncture a lymph node. Applicant also incorporates by reference herein, U.S. application Ser. No. 15/784,824 (U.S. Pub. No. 2018/0036514), and U.S. application Ser. No. 13/419,879 (U.S. Pub. No. 2012/0191180), both commonly owned herewith, for all that they disclose.

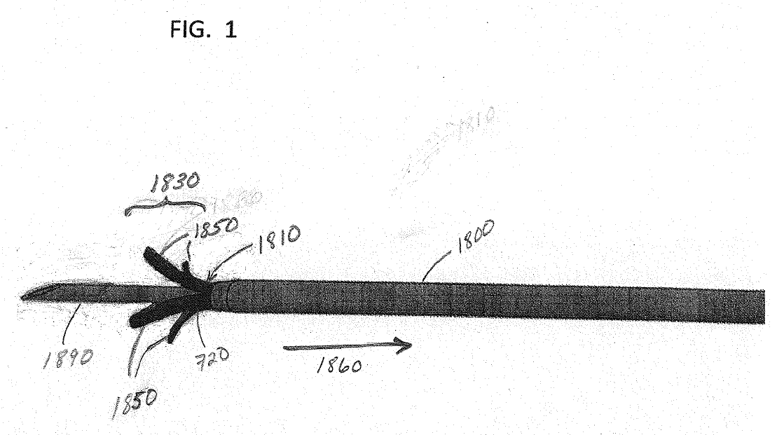

In a first embodiment, as shown in FIG. 1, an exemplary engagement catheter 1810 having a lymphatics needle 1890 for puncture access, and a flared stopper or stabilizer 1830, is shown. The engagement catheter 1810 comprises a flared stopper or stabilizer portion 1830 at its distal end 720 and is longitudinally surrounded by a retractable sleeve 1800. Although each of the engagement catheter 1810, sleeve 1800, and needle 1890 has a proximal end 710 and a distal end 720, FIG. 1 shows only the distal ends 720. Sleeve 1800 has a lumen through which engagement catheter 1810 has been slideably inserted, and engagement catheter 1810 has a lumen through which puncture needle 1890 has been slideably inserted. The sleeve 1800, engagement catheter 1810, and needle 1890 may each slide longitudinally relative to one another and are sized appropriately for puncture of a mammalian lymph node.

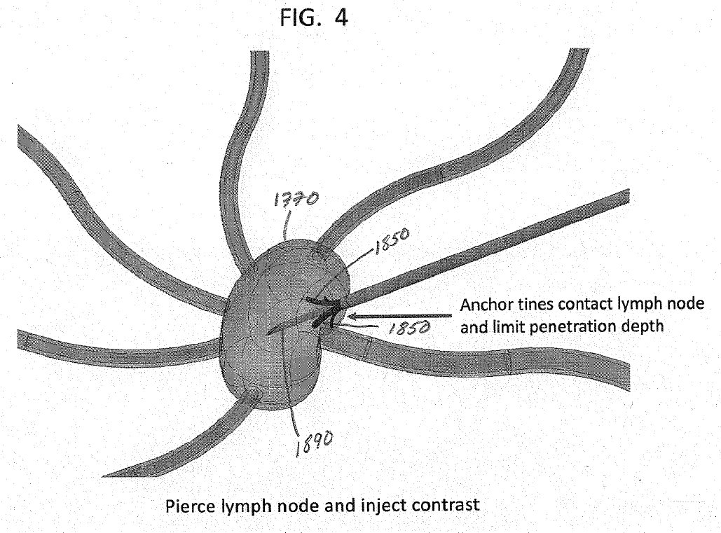

The engagement catheter 1810 has a flared stopper or stabilizer portion 1830 at its distal end to stabilize the engagement catheter 1810 relative to the lymph node 1770 to be punctured and to limit the penetration depth of needle 1890, as shown in FIG. 4. The flared stopper or stabilizer portion 1830 includes a plurality of flared tines 1850 at its distal end 720, as shown in FIG. 1, which contact the outer wall of the lymph node to prevent further advancement of the needle 1890. The engagement catheter 1810 may be formed as a hypo-tube with axially fenestrated (axially cut or slit) portions at its distal end 720. As shown in FIG. 1, these flared tines 1850 protrude or extend outward, away from the longitudinal axis of engagement catheter 1830. These flared tines 1850 may be 1-3 mm length curved protrusions, for example. As shown in FIG. 1, there may be four of the flared tines 1850. Alternatively, the engagement catheter 1810 may be axially fenestrated many more times to create more than four flared tines 1850. It should be understood that any shape, number, and size of flared tines 1850 may be used herein for the flared stopper or stabilizer 1830.

The lymphatic puncture needle 1890 may be a hollow, tapered, needle having a size 23-25G distal to 21-22G proximal, and a distal portion of 3-5 mm in length, for example. The needle 1890 may be hollow for delivering or injecting pharmaceuticals or contrast dyes, and/or may also be used under, or in combination with, vacuum/suction procedures to take biopsy samples, for example. In other embodiments, the lymphatics puncture needle 1890 may also be coated with and/or be formed of a material to aid in, or improve, ultrasound visualization, brightness, and/or contrast.

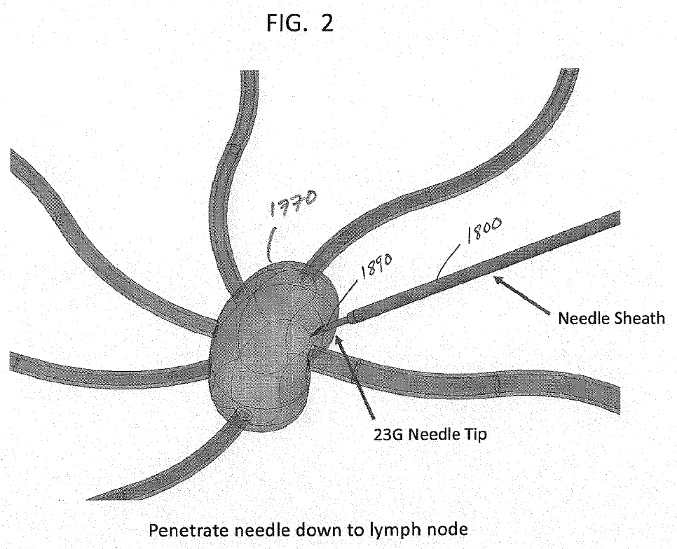

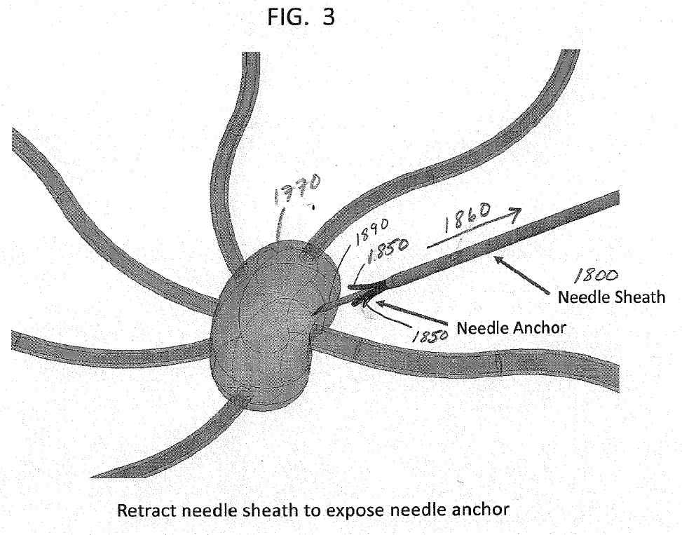

In operation, the tapered lymphatics puncture needle 1890 (having a distal portion of 3-5 mm in length) may be inserted under ultrasound guidance to approach the lymph node 1770, as shown in FIG. 2. To facilitate ultrasound visualization, the lymphatics puncture needle 1890 may also be coated and/or made of a material(s) which are easily seen/visable under ultrasound. Once the lymphatic puncture needle 1890 is in the desired position (relative to the lymph node), the needle sheath 1800 may be retracted toward its proximal end (in the direction of arrow 1860, as shown in FIGS. 1 and 3) to expose the flared stopper or stabilizer 1830, as shown in FIG. 3. The retraction of the sheath 1800 (in the direction of arrow 1860, as shown in FIGS. 1 and 3) may be accomplished by the physician using a handle portion (not shown) of the engagement catheter 1810. Once the sheath 1800 has been retracted and the tines 1850 of the flared stopper or stabilizer 1830 have been exposed, the needle 1890 may then be accelerated (i.e., pushed toward the lymph node) to puncture the lymph node 1770, as shown in FIG. 4.

As shown in FIG. 4, the flared tines 1850 of the stopper or stabilizer 1830 will contact the outer or exterior wall surface of the lymph node 1770 and prevent further advancement of the needle 1890 into the lymph node 1770. In this way, the flared tines 1850 of the stopper or stabilizer 1830 prevent over-perforation of the lymph node because only the 3-5 mm portion of the needle 1890 distal to the flared stopper or stabilizer 1830 can be advanced into the lymph node 1770. Once the needle 1890 punctures the lymph node 1770, a gentle pushing force (applied by the physician) on the needle 1890 (via an external handle, not shown) will maintain the stable position of the tip of the puncture needle 1890 and prevent loss of puncture access, while preventing further advancement (i.e., over-perforation) of the lymph node 1770.

The sleeve 1800 may be operated by a physician, using a handle portion or actuator portion located proximal to flared stopper or stabilizer 1830, to retract sleeve 1800 (in the direction of arrow 1860, as shown in FIGS. 1 and 3) and expose the flared tines 1850 of the stopper or stabilizer 1830. The handle portion may be part of a trans-septal access device or engagement catheter 1810, which remains partially outside of the patient's body during the medical procedure. In this embodiment, the physician may use an actuator positioned on the handle to retrace sleeve 1800. Alternatively, in another embodiment, the engagement catheter 1810 itself may be pushed toward proximal end 720 (in the direction opposite arrow 1860), so that the flared tines 1850 of stopper or stabilizer 1830 extend or protrude from the proximal end 720 of sleeve 1800.

While various embodiments of devices and systems and methods for using the same have been described in considerable detail herein, the embodiments are merely offered as non-limiting examples of the disclosure described herein. It will therefore be understood that various changes and modifications may be made, and equivalents may be substituted for elements thereof, without departing from the scope of the present disclosure. The present disclosure is not intended to be exhaustive or limiting with respect to the content thereof.

Further, in describing representative embodiments, the present disclosure may have presented a method and/or a process as a particular sequence of steps. However, to the extent that the method or process does not rely on the particular order of steps set forth therein, the method or process should not be limited to the particular sequence of steps described, as other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the scope of the present disclosure.

1. A device for puncturing a lymph node, comprising:
a sleeve having a proximal portion and a distal portion and a lumen extending between the proximal and distal portions;
an engagement catheter disposed within the lumen of the sleeve and having a proximal end and a distal end and a lumen extending between the proximal and distal ends;
a plurality of curved flared tines disposed at the distal end of the engagement catheter and flaring away from a longitudinal axis of the engagement catheter, wherein the sleeve and engagement catheter are configured to slide longitudinally relative to one another such that the plurality of curved flared tines disposed at the distal end of the engagement catheter are surrounded by the sleeve in a first position and the plurality of curved flared tines are operably deployed by retraction of the sleeve toward its proximal portion to a second position; and
a lymphatics puncture needle disposed within the lumen of the engagement catheter and operable to puncture a lymph node.

2. The device of claim 1, wherein the plurality of curved flared tines comprises four portions of the engagement catheter axially cut at its distal end.

3. The device of claim 1, wherein the plurality of curved flared tines comprises tines extending 1-3 mm.

4. The device of claim 1, wherein the lymphatics puncture needle comprises a tapered 23-25G distal to 21-22G proximal needle.

5. The device of claim 1, wherein the lymphatics puncture needle has a distal portion with a length of 3-5 mm, distal to a distal end of the plurality of curved flared tines when the plurality of curved flared tines have been deployed by retraction of the sleeve to the second position.

6. The device of claim 1, wherein the lymphatics puncture needle is coated with a material to aid in ultrasound visualization.

7. A method of puncturing a lymph node, comprising:
advancing a lymphatic puncture needle toward a lymph node;
retracing a sleeve to deploy a flared stabilizer;
advancing the lymphatic puncture needle to puncture the lymph node, until the flared stabilizer stops against an outer wall surface of a lymph node;
applying gentle pressure to the lymphatic needle to maintain a puncture site and stability without over-perforation of the lymph node.

8. The method of claim 7, wherein advancing the lymphatics puncture needle further comprises advancing the needle under ultrasound guidance.

9. The method of claim 7, wherein the flared stabilizer further comprises a plurality of tines extending outwardly from a center longitudinal axis of the flared stabilizer.

10. The method of claim 9, wherein the plurality of flared tines comprises four axially cut tines.

11. The method of claim 7, wherein retracting the sleeve comprises pulling the sleeve toward the operating physician.

12. The method of claim 7, wherein advancing the lymphatic puncture needle comprises advancing the needle 3-5 mm, until the flared stabilizer stops against an outer wall surface of a lymph node to prevent over-perforation of the lymph node.

13. The method of claim 7, wherein retracting the sleeve comprises operating an actuator located on a handle of a catheter.
